(19) United States
(12) Patent Application Publication
(10) Pub. No.: US 2015/0083128 A1
(43) Pub. Date: Mar. 26, 2015

(54) DEVICE FOR DISPENSING FLUID PRODUCT

(71) Applicant: APTAR FRANCE SAS, Le Neubourg (FR)

(72) Inventors: Matthieu Baillet, Bonsecours (FR); Maxime Kirniak, Rouen (FR); Antoine Laut, Wy Dit Joli Village (FR)

(73) Assignee: APTAR FRANCE SAS, Le Neubourg (FR)

(21) Appl. No.: 14/396,186

(22) PCT Filed: May 30, 2013
(86) PCT No.: PCT/FR2013/051217

§ 371 Date: Oct. 22, 2014

(30) Foreign Application Priority Data
May 31, 2012 (FR) 1255011

(51) Int. Cl. A61M 15/00

(52) U.S. Cl. CPC A61M 15/004; A61M 15/0021; A61M 15/0051; A61M 15/0055; A61M 15/0071; A61M 15/0086; A61M 2202/064; A61M 2205/19
USPC 128/203.15

(57) ABSTRACT

A fluid dispenser device including a main body (10), said device including at least one individual reservoir containing a single dose of fluid, such as powder, opening means (80) being provided for opening an individual reservoir each time the device is actuated, an upper body (101) being mounted in stationary manner on said main body (10), said upper body (101) receiving a mouthpiece (200) that defines a dispenser orifice, said device including an inhalation trigger system (60) that comprises a deformable air chamber (61) that co-operates with said inhalation piece (200), and a trigger element (600) that co-operates with said air chamber (61), such that during inhalation through said inhalation piece (200), said air chamber (61) is deformed and said trigger element (600) actuates said opening means (80), such that during inhalation through the inhalation piece (200), a reservoir is opened by said opening means, the fluid dispenser device being characterized in that said mouthpiece (200) is fastened to said upper body (101), said mouthpiece including a deformable peripheral lip (209) that, after assembly, co-operates in leaktight manner with a peripheral edge (108, 109) of said upper body (101).

The present invention relates to a fluid dispenser device, and more particularly to a dry-powder inhaler.

Inhalers are well known in the prior art. Various types exist. A first type of inhaler contains a reservoir receiving many doses of powder, the inhaler being provided with metering means making it possible, on each actuation, to remove one dose of said powder from the reservoir, so as to bring said dose into an expulsion duct in order to be dispensed to the user. Inhalers including individual reservoirs, such as capsules, that are loaded into the inhaler just before said reservoir is used are also described in the prior art. The advantage of such devices is that it is not necessary to store all of the doses inside the appliance, such that said appliance can be compact. However, the inhaler is more difficult to use, since the user is obliged to load a capsule into the inhaler before each use. Another type of inhaler consists in placing the doses of powder in individual predosed reservoirs, then in opening one of the reservoirs each time the inhaler is actuated. That implementation seals the powder more effectively since each dose is opened only when it is about to be expelled. In order to make such individual reservoirs, various techniques have already been proposed, such as an elongate blister strip or blisters disposed on a rotary circular disk. All existing types of inhalers, including those described above, present both advantages and drawbacks associated with their structures and with their types of operation. Thus, with certain inhalers, there is the problem of metering accuracy and reproducibility on each actuation. In addition, the effectiveness of the dispensing, i.e. the fraction of the dose that effectively penetrates into the user's lungs in order to have a beneficial therapeutic effect, is also a problem that exists with a certain number of inhalers. A solution for solving that specific problem has been to synchronize the expulsion of the dose with the inhalation of the patient. Once again, that can create drawbacks, in particular in that type of device, the dose is generally initially loaded into an expulsion duct before inhalation, then expulsion is synchronized with inhalation. That means that if the user drops, shakes, or manipulates the inhaler in an undesirable or inappropriate manner between the moment when the user loads the dose (either from a multidose reservoir or from an individual reservoir) and the moment when the user inhales, then the user risks losing all or part of the dose, with said dose possibly being spread about inside the appliance. In that event, there can exist a high risk of overdosing the next time the device is used. The user who realizes that the dose is not complete will load a new dose into the appliance, and while the new dose is being inhaled, a fraction of the preceding dose that was lost in the appliance could thus be expelled at the same time as the new dose, thereby causing an overdose. In the treatments envisaged, such overdosing can be very harmful, and the authorities in all countries are issuing ever-stricter requirements to limit the risk of overdosing as much as possible. Another problem that may occur relates to assembling certain parts, in particular movable parts, that need to withstand large stresses in operation, and for which assembly needs to be particularly reliable so as to avoid any risk of malfunctioning. With the small size of certain parts and possible deformations resulting from molding and/or from conditioning plastic components, it can be complicated to guarantee such reliable assembly. Leaktight assemblies are desirable, in particular in inhalers using an inhalation trigger system, in particular so as to avoid any head loss during inhalation, and also so as to ensure that the trigger threshold is repeatable. This improves comfort in use for patients, and makes it possible to avoid disturbing the habits of patients in the event of variations from one inhaler to another. Documents U.S. Pat. No. 6,179,164, FR-2 936 425, and WO 02/085281 describe prior-art devices.

An object of the present invention is to provide a fluid dispenser device, in particular a dry-powder inhaler, that does not have the above-mentioned drawbacks.

In particular, an object of the present invention is to provide such a device that is simple and inexpensive to manufacture and to assemble, that can be assembled and used reliably, guaranteeing metering accuracy and reproducibility on each actuation, providing an optimum yield with regard to the effectiveness of the treatment, by making it possible to dispense a substantial fraction of the dose to the zones to be treated, in particular the lungs, avoiding, in safe and effective manner, any risk of overdosing, and that is as compact as possible, while guaranteeing sealing and absolute integrity of all of the doses up to their expulsion.

The present invention thus provides a fluid dispenser device including a main body, said device including at least one individual reservoir containing a single dose of fluid, such as powder, opening means being provided for opening an individual reservoir each time the device is actuated, an upper body being mounted in stationary manner on said main body, said upper body receiving a mouthpiece that defines a dispenser orifice, said device including an inhalation trigger system that comprises a deformable air chamber that co-operates with said inhalation piece, and a trigger element that co-operates with said air chamber, such that during inhalation through said inhalation piece, said air chamber is deformed and said trigger element actuates said opening means, such that during inhalation through the inhalation piece, a reservoir is opened by said opening means, said mouthpiece being fastened to said upper body, said mouthpiece including a deformable peripheral lip that, after assembly, co-operates in leaktight manner with a peripheral edge of said upper body.

Advantageously, said peripheral edge of the upper body is beveled and comprises a top edge portion and a bottom edge portion.

Advantageously, the slopes of said top and bottom edge portions are different.

Advantageously, said mouthpiece includes snap-fastener slots that are adapted to snap-fasten on snap-fastener projections of said upper body.

Advantageously, each snap-fastener slot includes a deformable tab that is deformed by a corresponding snap-fastener projection during assembly.

Advantageously, said upper body includes positioner lugs that, during assembly, are adapted to co-operate with positioner housings of the mouthpiece.

Advantageously, the device includes movable support means that are adapted to move an individual reservoir against said opening means on each actuation, said movable support means being displaceable between a non-dispensing position and a dispensing position, said movable support means being urged towards their dispensing position by resilient means, such as a spring or a spring blade, and being held in their non-dispensing position by blocking means that are released by the user inhaling.

Advantageously, said opening means include a perforator element that is stationary relative to said main body and that is adapted to cut a closure wall of the reservoir in such a manner that the cut portion(s) does/do not obstruct the opening(s) that is/are formed.

Advantageously, the device includes at least one cover element that is mounted to pivot on said main body between a closed position and an open position.

These characteristics and advantages and others of the present invention appear more clearly from the following detailed description, given by way of non-limiting example, and with reference to the accompanying drawing, and in which:

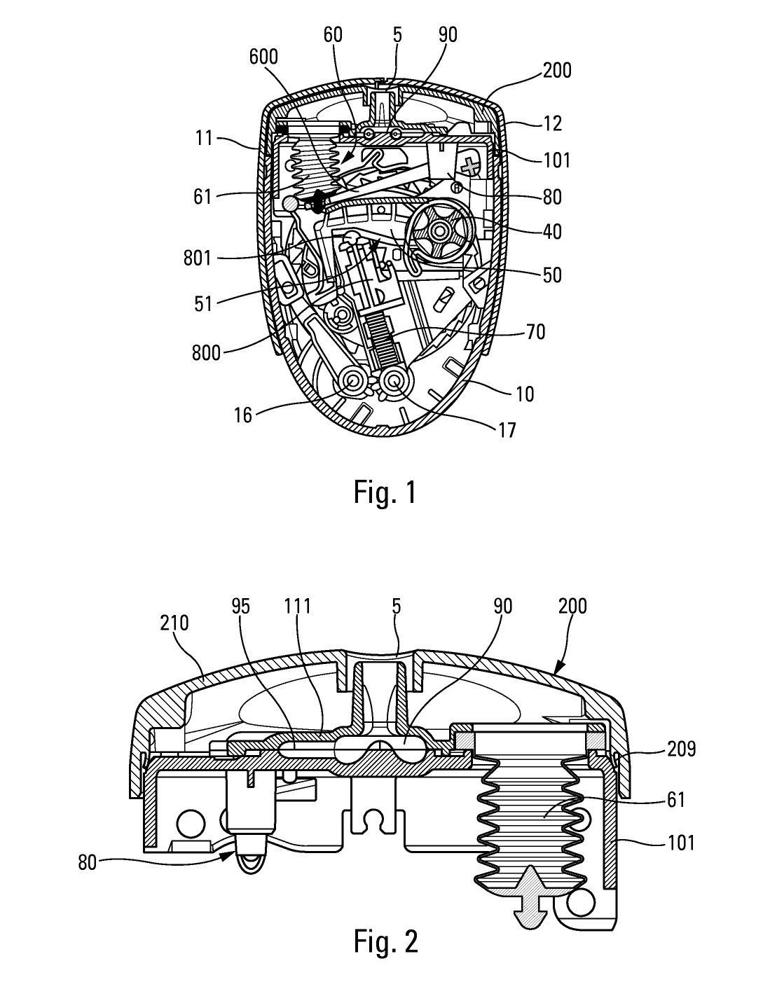

FIG. 1 is a diagrammatic section view of a dispenser device in an advantageous embodiment of the invention, in its rest position;

FIG. 2 is a diagrammatic view of a detail of a portion of the FIG. 1 device;

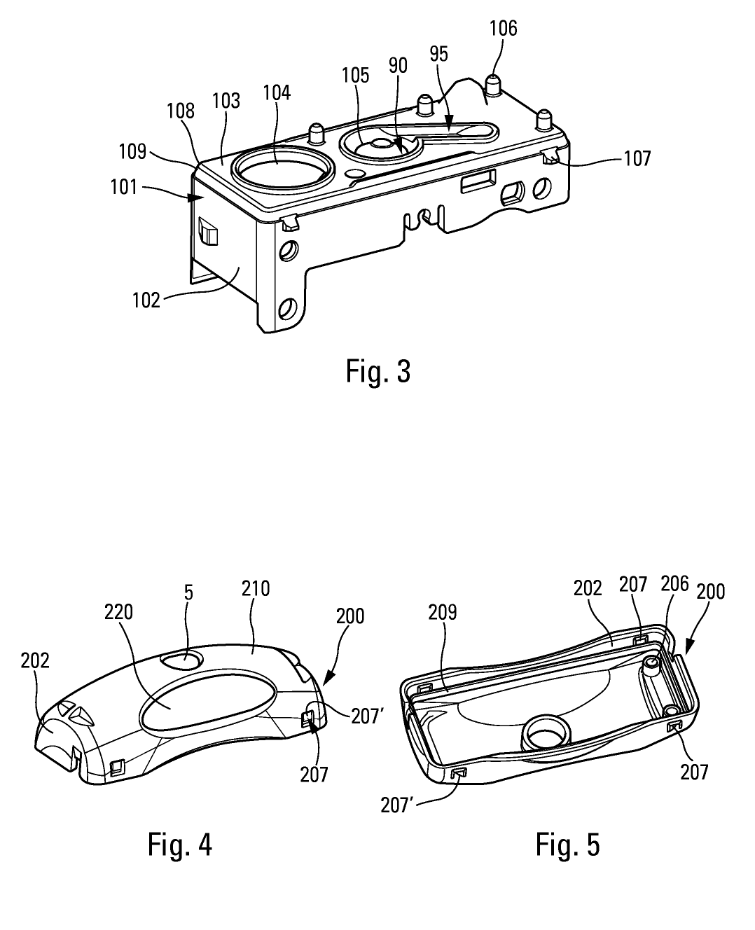

FIG. 3 is a diagrammatic perspective view of the upper body;

FIGS. 4 and 5 are diagrammatic perspective views of the mouthpiece, from above and below respectively; and

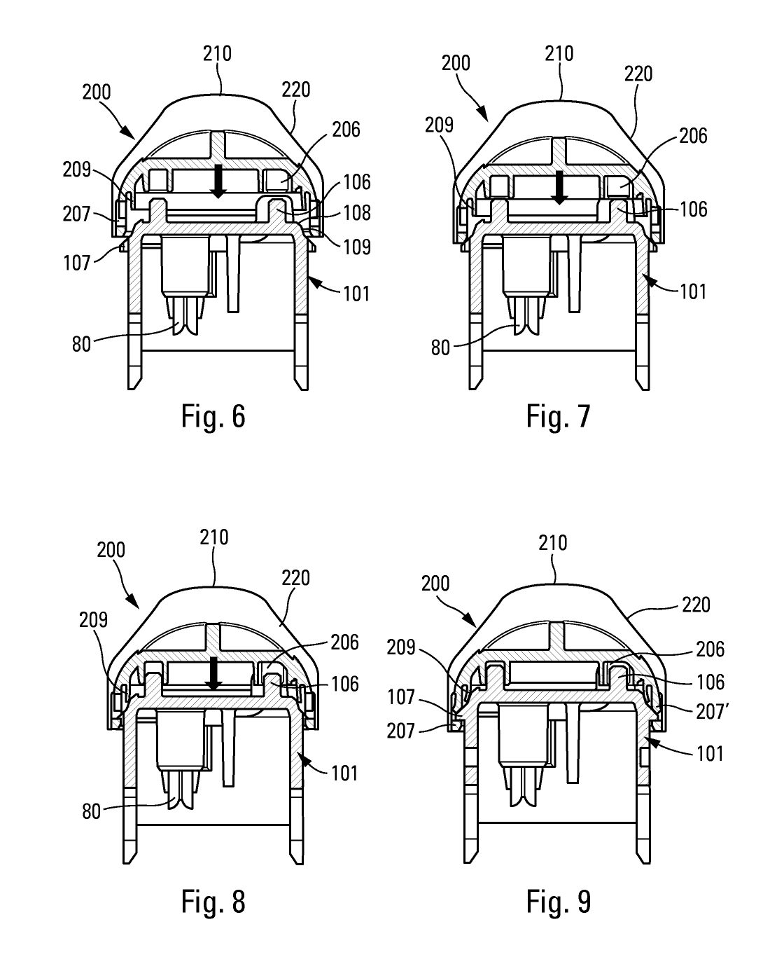

FIGS. 6 to 9 are diagrammatic and fragmentary cross-section views showing the assembly stage for assembling the mouthpiece on the upper body.

FIGS. 1 and 2 show an advantageous embodiment of a dry-powder inhaler. The inhaler includes a main body 10 on which there can be slidably mounted two cover-forming portions 11, 12 that are adapted to be opened so as to open and prime the device. The main body 10 can be approximately rounded in shape, as shown in the figures, but it could be of any other appropriate shape. An upper body 101 is assembled to the main body 10, and a mouthpiece 200 is assembled on said upper body 101. The mouthpiece 200 defines a dispenser orifice 5 through which the user inhales while the device is being actuated. The dispenser orifice 5 is typically arranged approximately in the center of the mouthpiece 200. The covers 11, 12 can open by pivoting about a common pivot axis, or about two parallel axes by being meshed together. Any other opening means for opening the device can be envisaged. In a variant, the device could include only a single cover instead of two.

Inside the main body 10 there is provided a strip (not shown for the sake of clarity) of individual reservoirs, also known as blisters, said strip being made in the form of an elongate strip on which the blisters are disposed one behind another, in manner known per se. The blister strip is advantageously constituted by a base layer or wall that forms the cavities receiving the doses of powder, and by a closure layer or wall that covers each of said blisters in sealed manner. Before first use, the blister strip can be rolled-up inside the main body 10, preferably in a storage portion, and first strip displacement means 40, in particular rotary means, are provided for progressively unrolling the blister strip and for causing it to advance.

Second displacement means 50, in particular means that are mounted to pivot on the main body 10, are provided for bringing a respective blister into a dispensing position each time the device is actuated. The second displacement means are advantageously mounted to pivot between a non-dispensing position and a dispensing position in which a blister co-operates with said opening means.

The strip portion including the empty blisters is advantageously adapted to be rolled-up at another location of said body 10, preferably a reception portion, as described in greater detail below.

The inhaler includes blister opening means 80 preferably comprising a perforator and/or cutter needle for perforating and/or cutting the closure layer of the blisters. Preferably, the opening means comprise a perforator element 80 that is stationary relative to the main and upper bodies 10, 101, and against which a respective blister is displaced on each actuation by the second displacement means. The blister is thus perforated by said perforator element that penetrates into said blister so as to expel the powder by means of the user inhaling. Advantageously, the perforator element is adapted to cut a closure wall of the reservoir in such a manner that the cut portion(s) does/do not obstruct the opening(s) that is/are formed. Documents WO 2006/079750 and WO 2009/007640 describe such blister opening means, and they are thus incorporated in the present description by way of reference.

The first displacement means 40 are adapted to cause the blister strip to advance after each inhalation of the user. The second displacement means 50 are adapted to displace the blister to be emptied against said opening means during actuation, before each inhalation. The second displacement means can be urged by a resilient element 70, such as a spring or any other equivalent resilient element, said resilient element being suitable for being prestressed while the device is being opened.

Preferably, the first displacement means 40 are formed by an indexer wheel that receives and guides the blister strip. The description below is thus made with reference to such an indexer wheel 40. Turning the indexer wheel 40 causes the blister strip to advance. Before each inhalation, a full blister is always in a position facing the opening means 80. The second displacement means 50 can include a pivot member that is mounted to pivot about a pivot axis, said indexer wheel 40 advantageously being rotatably mounted on said pivot member.

An actuation cycle of the device can be as follows. During opening of the device, the two cover-forming lateral portions 11, 12 are moved away from each other by pivoting about the body so as to open the device and thus spring-load the device. In this position, the indexer wheel 40 cannot be displaced towards the perforator element 80, since the second displacement means 50 are held by appropriate blocking means (not shown for the sake of clarity). Documents WO 2009/077700 and WO 2009/136098 describe such blocking means, and they are thus incorporated in the present description by way of reference. While the user is inhaling through the mouthpiece, the blocking means are unblocked, thereby causing said indexer wheel 40 to move towards the needle, and thereby causing a blister to be opened.

As explained above, it is desirable for the opening means to be actuated by the user inhaling. In order to trigger the opening means by inhalation, an inhalation trigger system 60 is provided that advantageously comprises an air chamber 61 that is deformable under the effect of inhalation, the air chamber being adapted to release the blocking means. The air chamber 61 may advantageously be made in the form of a bellows. Inhalation by the user causes said deformable air-chamber to deform, thereby releasing said blocking means and enabling the second displacement means to be displaced, and therefore enabling a respective blister to be displaced towards its opening position. The blister is therefore opened only on inhalation, such that it is emptied simultaneously. Thus, there is no risk of any of the dose being lost between opening the blister and emptying it.

The inhaler further includes a dispersion chamber 90 for receiving the dose of powder after a respective blister has been opened. The dispersion chamber is advantageously provided with at least one and preferably more beads that are displaced inside said chamber 90 during inhalation, in particular so as to improve dispensing of the air and powder mixture after a blister has been opened, in order to increase the effectiveness of the device.

It can be advantageous for the opening means, in particular for the perforator element, to be connected directly to said dispersion chamber, e.g. via a channel 95 leading to said chamber 90.

After inhalation, when the user closes the device, all of the components return to their initial, rest positions. The device is thus ready for a new utilization cycle.

In an advantageous aspect of the inhaler, the blisters are formed on a flexible elongate strip that, initially, is mainly stored in the form of a roll in a storage housing inside the main body 10 of the device. Advantageously, the rolled-up blister strip is held by inner walls of said storage housing without its rear end (rear in the advancement direction of the blister strip) being fastened relative to said main body 10, thereby enabling the blister-strip roll to be assembled more easily inside the device. The blister strip is displaced by means of the indexer wheel 40 that advantageously presents at least one and preferably more recesses, each having a shape that corresponds to the shape of the blisters. Thus, when the indexer wheel 40 turns, it causes the blister strip to advance. Naturally, in a variant or in additional manner, it is possible to use other means for advancing the blister strip, e.g. providing a profile on the longitudinal lateral edges of the blister strip, said profile being adapted to co-operate with appropriate drive means. In addition, holes formed along the lateral edges of the blister strip could also be used to cause the blister strip to advance by means of sprocket wheels co-operating with said holes.

After opening one or more blisters, the blister-strip portion with the empty blisters must be suitable for being stored in easy and compact manner in the device, while avoiding any risk of blockage. Advantageously, the used blister strip is rolled-up automatically, once again forming a roll.

In still another aspect of the inhaler, a dose counter or indicator device (not shown for the sake of clarity) may also be provided. The device may include numbers or symbols that are marked directly on the blister strip, and that are visible through an appropriate window in the main body 10 of the device. In a variant, it is possible to envisage using a counter with one or more rotary disks or rings including numbers or symbols. Documents WO 2008/012458 and WO 2011/154659 describe such counters, and they are thus incorporated in the present description by way of reference. An object of the invention is to avoid counting doses that have not been dispensed, e.g. in the event of a manipulation error, or of an incomplete manipulation of the device. It is thus desirable that the counter or indicator is actuated only once the user has inhaled, since it is this inhalation that makes it possible for the blister to open and the dose contained therein to be dispensed. Advantageously, the counter is thus actuated after inhalation, when the user closes the device.

The movable cover element 12 is secured to a cocking member 800 that can slide in an appropriate housing. The cocking member 800 thus advantageously pivots relative to said body 10 together with the cover element 12. The cocking member 800 may be moved against a spring 70, advantageously a coil spring, that is arranged in said housing. The cocking member 800 is thus connected at one end to said spring 70, and at the other end it co-operates with the second displacement means, in particular with a pivot member 50 that is mounted to pivot on the body 10, and on which the indexer wheel 40 is fastened is rotary manner.

When the movable cover element 12 is opened, the cocking member 800 is displaced in its housing, compressing the spring 70. The pivot member 50 of the second displacement means is itself prevented from moving by the above-mentioned blocking means that are released only at the moment of inhalation. Thus, in the absence of any inhalation in the open position, closing the cover elements 11, 12 would merely cause the cocking member 800 to return to its rest position and the spring 70 to decompress.

Thus, by opening the inhaler, the user primes the system. If the user does not inhale and closes the inhaler, said inhaler merely returns to its start position without displacing the blister strip or the blocking means. There is thus no risk of a blister (and thus an active dose of substance) being lost by accidental or incomplete actuation in which the user does not inhale between opening and closing. Opening the blister, emptying it, dispensing the powder into the lungs of the user, displacing the blister strip to bring a new full blister to face the opening means, and counting the dose are thus possible only if the user inhales.

The blocking means that block the second displacement means and in particular the pivot member that co-operates with the cocking member, are connected to the deformable air chamber 61 that is sensitive to the user inhaling, via a trigger element 600, so that while the user is inhaling, said deformable air chamber deforms, causing the trigger element 600 to pivot and causing said blocking means to be released. This enables said second displacement means to be displaced towards their dispensing position under the effect of the force exerted by the compressed spring 70 on the cocking member 800 that pushes against the pivot member 50. Such displacement causes a full blister to be opened and a dose to be dispensed.

A cam surface 51 is formed on said movable support means 50, on which the cocking member 800 slides. The cocking member 800 is thus adapted to compress the spring 70 when the cover element 12 is open, and to decompress said spring 70 when said cover element 12 is closed.

Advantageously, in its portion in contact with the cam surface 51, the cocking member 800 includes a rounded portion 801 for facilitating sliding of the cocking member 800 on said cam surface 51.

In this embodiment, the movable support means are made in the form of a member 50 that is pivotally mounted on the body 10 about a pivot axis. Since the above-mentioned cam surface 51 is formed on said pivot member 50, when the spring 70 is loaded while the movable cap element 12 is opening, said pivot member 50 is urged towards its dispensing position by said cocking member 800 and the spring 70 is compressed.

The cam surface 51 may include at least two portions of different slopes that are advantageously separated by a vertex. Starting once again from the closed position of the movable cover element, the first slope portion on which the cocking member 800 slides, enables the spring 70 to be compressed, as described above. When the spring is loaded, i.e. compressed, the cam surface 51 may provide a second different slope portion with which the cocking member 800 co-operates only when the device is in its open position. The cocking member 800 preferably exerts a force that is substantially perpendicular on the second cam surface portion. In this way, the loaded position is stable. In a variant, the second slope portion forms an abutment notch in which the cocking member 800 comes to be positioned in the open position.

After inhalation, i.e. in the dispensing position, the blocking means have been released, and the movable support means 50 have been displaced upwards by the compressed spring 70.

Advantageously, the two movable cover elements 11, 12 mesh together via appropriate gearing so as to guarantee symmetrical opening and closing of said two movable cover elements. They can mesh together in the proximity of their pivot axes 16, 17.

FIG. 3 shows more particularly the upper body 101, which comprises a peripheral side wall 102 that is fastened to the main body 10, and a top wall 103 that is substantially plane. In the embodiment in FIG. 3, the side wall 102 is approximately rectangular in shape, but other shapes are also possible. The top wall 103 includes an opening 104 for receiving the deformable air chamber 61 of the inhalation trigger system 60. The top wall 103 also includes a recess 105 that defines the bottom wall of the dispersion chamber 90 and of the associated channel 95. A cover portion 111 is for covering said recess 105, as can be seen in FIG. 2. The top wall 103 also includes positioner lugs 106 for co-operating with corresponding positioner housings provided in the bottom wall of the mouthpiece, as described below. At the junction between the side wall 102 and the top wall 103, a peripheral edge is provided. The peripheral edge is preferably beveled and advantageously includes a top edge portion 108 in contact with the top wall 103, and a bottom edge portion 109 in contact with the side wall 102. Advantageously, the slopes of the top and bottom edge portions may be different. The upper body 101 further includes snap-fastener projections 107 that are adapted to co-operate with corresponding snap-fastener slots of the mouthpiece, so as to fasten the mouthpiece on the upper body, as described below. The snap-fastener projections 107 are advantageously arranged on the side wall 102, level with the bottom edge portion 109.

FIGS. 4 and 5 show more particularly the mouthpiece 200. In general, the mouthpiece 200 is preferably symmetrical about said dispenser orifice 5. It comprises a peripheral side wall 202 having a shape that matches the shape of the peripheral side wall 102 of the upper body 101, and a top wall 210. The top wall 210 extends longitudinally on both sides of the dispenser orifice 5. It should be observed that the top wall 210 is also slightly humped or convex in the longitudinal direction, thereby imparting a flattened rounded shape to the mouthpiece 200. On either side of the dispenser orifice 5, in the transverse or lateral direction, the mouthpiece includes two lateral bearing surfaces 220 that are respectively provided laterally on each side of said dispenser orifice 5. Each of the lateral bearing surfaces 220 is for receiving a lip of the user during inhalation. The lateral bearing surfaces 220 are slightly concave, in particular in the transverse direction, so as to form slight hollows that are adapted to position the lips of the user properly, when said user places them on the mouthpiece. As can be seen in particular in FIG. 4, the lateral bearing surfaces 220 extend said top wall 210 laterally, and they are preferably spaced apart from each other by the top wall 210 and by the dispenser orifice 5. In this way, when the user places the mouth on the mouthpiece 200, the spacing between the two lateral bearing surfaces 220 obliges the user to open the mouth wide enough to avoid the teeth obstructing the dispenser orifice 5, even in part, during inhalation. In addition, the slightly concave shape of the lateral bearing surfaces 220 is ergonomic, ensuring good sealing with the mouth of the user on the mouthpiece 200 during inhalation. In particular, this avoids head loss during inhalation, and guarantees that as much as possible of the user-created inhalation flow is used to actuate the inhaler and to expel the powder contained in the inhaler. The spacing and the depth of the lateral bearing surfaces 220 also guarantee that the dispenser orifice is positioned inside the mouth, beyond the teeth, but without the dispenser orifice 5 being formed on a projecting portion of the mouthpiece 200.

The lateral bearing surfaces 220 advantageously extend longitudinally over a central portion of said mouthpiece 200, preferably over the major part thereof, as shown in particular in FIG. 4. Advantageously, the two lateral bearing surfaces 220 are completely symmetrical to each other, about the dispenser orifice 5.

With reference once again to FIG. 1, it should be observed that the cover elements 11, 12 that are movable about the main body 10 of the inhaler and about the mouthpiece 200, include a top surface of convex shape that is substantially adapted to the shape of the mouthpiece, in particular at its top wall 210. In this way, while said cover elements are being displaced between the open and closed positions, no gap is created as a result of the presence of the mouthpiece 200, such that the fingers of the user do not risk being pinched while manipulating the cover elements. In general, the mouthpiece does not include any projecting portion that risks creating a gap that could receive the user's fingers while the device is being actuated.

The side wall 202 of the mouthpiece 200 includes the above-mentioned snap-fastener slots 207 that snap-fasten on the snap-fastener projections 107 of the upper body while the mouthpiece is being assembled on said upper body. Advantageously, there are four slots 207 for four projections 107. Advantageously, each snap-fastener slot 207 may include a deformable tab 207′ that is deformed, in particular radially outwards, by a respective projection, as can be seen in FIG. 9.

On its inside surface, the top wall 210 includes the positioner housings 206 that are adapted to co-operate with the positioner lugs 106 while the mouthpiece is being assembled. This guarantees that the mouthpiece 200 is positioned accurately relative to the upper body 101 during assembly.

In the invention, the mouthpiece 200 includes a deformable inner peripheral lip 209 that is adapted to co-operate with the beveled edge 108, 109 of the upper body 101. The lip 209 deforms during assembly so as to improve the sealing of the assembly.

Advantageously, during assembly, the lip 209 co-operates initially with the top edge portion 108 that is radially further in and with a slope that is smaller (relative to the horizontal in the position in FIGS. 2 and 6 to 9). This guarantees that the lip 209 is guided properly over the bottom edge portion 109 where leaktight contact is made after assembly.

FIGS. 6 to 9 show the assembly stage. In FIG. 6, the mouthpiece 200 is positioned above the upper body 101 and moves downwards in the direction of the arrow. In FIG. 7, the positioner lugs 106 co-operate with the positioner housings 206 so as to align the mouthpiece 200. As assembly continues, the lugs 106 penetrate progressively into the respective housings. Simultaneously, the bottom edge of the snap-fastener slots 207 come into contact with the snap-fastener projections 207, and the lip 209 comes into contact with the top edge portion 108, as can be seen in FIG. 8.

After assembly, as shown in FIG. 9, the lugs 106 are in the housings 206, the slots 207 are snap-fastened on the projections 107, with the deformable tabs 207′ deformed, and the lip 209 is in leaktight peripheral contact with the bottom edge portion 109.

Advantageously, the deformable tabs 107′ guarantee that the mouthpiece 200 is positioned under stress on the upper body 101 in stable and repeatable manner. Thus, the lip 209 has the sole function of guaranteeing sealing. This has the advantage of offering independent ways of optimizing functions, e.g. optimizing the quality of positioning without risk of degrading the sealing function.

The shapes, dimensions, and positions of the slots 207, projections 107, and deformable tabs 207′ may be adjusted so as to optimize assembly. The lip 209 may have a profile that is appropriate for optimizing its sealing co-operation with the peripheral edge of the upper body.

The present invention therefore makes it possible to provide a dry-powder inhaler that provides the following features:
a plurality of individual doses of powder stored in individual sealed blisters, e.g. 30 or 60 doses stored on a rolled-up strip;
the powder is released by perforation that is achieved by the user inhaling, the blister being perforated by means of an inhalation detector system that is coupled to a prestressed release system;
appropriately-shaped drive means that are engaged with blisters so as to displace the blister strip after each inhalation, and bring a new full blister into a position in which it is to be opened by appropriate opening means;
means for avoiding doses being lost in the event of the inhaler being opened, but in the absence of any inhalation; and
a dose indicator adapted to count the doses only in the event of inhalation.

Other features are also provided by the device of the invention as described above.

It should be observed that the various features, even if they are shown as being provided simultaneously on the inhaler, could be implemented separately. In particular, the inhalation trigger mechanism could be used regardless of the type of reservoir opening means, regardless of the use of a dose indicator, regardless of the way in which the individual blisters are arranged relative to one another, etc. The cocking means and the inhalation trigger system could be made in some other way. The same applies for other component parts of the device.

Various modifications are also possible for the skilled person without departing from the scope of the present invention as defined in the accompanying claims. In particular, the various characteristics and functions of the device described with reference to the drawings can be combined together in any appropriate manner.

1-11. (canceled)

12. A fluid dispenser device including a main body, said device including at least one individual reservoir containing a single dose of fluid, such as powder, opening means being provided for opening an individual reservoir each time the device is actuated, an upper body being mounted in stationary manner on said main body, said upper body receiving a mouthpiece that defines a dispenser orifice, said device including an inhalation trigger system that comprises a deformable air chamber that co-operates with said inhalation piece, and a trigger element that co-operates with said air chamber, such that during inhalation through said inhalation piece, said air chamber is deformed and said trigger element actuates said opening means, such that during inhalation through the inhalation piece, a reservoir is opened by said opening means, wherein said mouthpiece is fastened to said upper body, said mouthpiece including a deformable peripheral lip that, after assembly, co-operates in leaktight manner with a beveled peripheral edge of said upper body.

13. A device according to claim 12, wherein said beveled peripheral edge comprises a top edge portion and a bottom edge portion.

14. A device according to claim 13, wherein the slopes of said top and bottom edge portions are different.

15. A device according to claim 14, wherein said top edge portion is radially further in and has a smaller slope compared to said bottom edge portion.

16. A device according to claim 15, wherein, during assembly, said deformable lip co-operates initially with said top edge portion, so as to guarantee that said deformable lip is guided over said bottom edge portion where sealing is achieved after assembly.

17. A device according to claim 12, wherein said mouthpiece includes snap-fastener slots that are adapted to snap-fasten on snap-fastener projections of said upper body.

18. A device according to claim 17, wherein each snap-fastener slot includes a deformable tab that is deformed by a corresponding snap-fastener projection during assembly.

19. A device according to claim 12, wherein said upper body includes positioner lugs that, during assembly, are adapted to co-operate with positioner housings of the mouthpiece.

20. A device according to claim 12, including movable support means that are adapted to move an individual reservoir against said opening means on each actuation, said movable support means being displaceable between a non-dispensing position and a dispensing position, said movable support means being urged towards their dispensing position by resilient means, such as a spring or a spring blade, and being held in their non-dispensing position by blocking means that are released by the user inhaling.

21. A device according to claim 12, wherein said opening means include a perforator element that is stationary relative to said main body and that is adapted to cut a closure wall of the reservoir in such a manner that the cut portion(s) does/do not obstruct the opening(s) that is/are formed.

22. A device according to claim 12, including at least one cover element that is mounted to pivot on said main body between a closed position and an open position.
